(19) United States
(12) Patent Application Publication
(10) Pub. No.: US 2025/0169811 A1
(43) Pub. Date: May 29, 2025

(54) TENSIONABLE TISSUE REPAIR SYSTEMS AND SURGICAL METHODS

(71) Applicant: ARTHREX, INC., Naples, FL (US)

(72) Inventors: Thomas DOONEY, Naples, FL (US); Peter J. DREYFUSS, Naples, FL (US)

(21) Appl. No.: 18/954,720

(22) Filed: Nov. 21, 2024

Related U.S. Application Data
(60) Provisional application No. 63/603,257, filed on Nov. 28, 2023.

(51) Int. Cl. A61B 17/04; A61F 2/08

(52) U.S. Cl. CPC A61B 17/0401; A61B 2017/0404; A61B 2017/0464; A61F 2/0811; A61F 2002/0852

(57) ABSTRACT

Tensionable tissue repair systems and methods are provided for reducing and fixating tissue to bone. The proposed systems and methods utilize one or more suture anchors and one or more surgical buttons for reducing and fixating the tissue to the bone. Additional suture anchors may be used as part of a multi-row fixation technique for providing a desired area of footprint compression over top of the tissue.

### CROSS-REFERENCE TO RELATED APPLICATIONS

This application claims the benefit of U.S. Provisional Application No. 63/603,257, which was filed on Nov. 28, 2023 and is incorporated herein by reference in its entirety.

### BACKGROUND

This disclosure relates to the field of surgery, and more particularly to systems and associated surgical methods for tensioning, fixating, and compressing tissue relative to bone.

Repetitive trauma to a joint, such as a knee, ankle, hip, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to tensionable tissue repair systems and methods for reducing and fixating tissue to bone. The proposed systems and methods utilize one or more suture anchors and one or more buttons for reducing and fixating the tissue to the bone. Additional suture anchors may be used as part of a multi-row fixation technique for providing a desired area of footprint compression over top of the tissue.

An exemplary surgical method may include, inter alia, inserting a first suture anchor into a bone, passing a first suture of the first suture anchor through a tissue, feeding the first suture through a first attachment section of a surgical button, securing the first suture relative to the surgical button, inserting a second suture anchor into the bone at a position that is lateral to the first suture anchor, feeding a second suture of the second suture anchor through a second attachment section of the surgical button, and securing the second suture relative to the surgical button. The second attachment section is thinner and/or less dense than the first attachment section of the surgical button.

Another exemplary surgical method may include, inter alia, implanting a first suture anchor into a bone. A first suture of the first suture anchor extends through a tissue and is attached to a first attachment section of a surgical button. The method further includes tensioning the first suture to fixate the surgical button against the tissue, inserting a second suture anchor into the bone at a position that is lateral to the first suture anchor, passing a second suture of the second suture anchor through a second attachment section of the surgical button, and securing the second suture back to the second suture anchor.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 schematically illustrates a step of surgical method for reattaching torn tissue to bone.

FIG. 2 illustrates another step of a surgical method for reattaching torn tissue to bone.

FIGS. 3 and 4 illustrate an exemplary knotless suture anchor.

FIGS. 5 and 6 illustrate another exemplary knotless suture anchor.

FIG. 7 illustrates another step of a surgical method for reattaching torn tissue to bone.

FIG. 8 illustrates another step of a surgical method for reattaching torn tissue to bone.

FIG. 9 illustrates another step of a surgical method for reattaching torn tissue to bone.

FIG. 10 illustrates another step of a surgical method for reattaching torn tissue to bone.

FIG. 11 illustrates another step of a surgical method for reattaching torn tissue to bone.

FIG. 12 illustrates yet another step of a surgical method for reattaching torn tissue to bone.

FIG. 13 illustrates a final repair construct of a surgical method for reattaching tissue to bone.

FIGS. 14 and 15 illustrate yet another exemplary knotless suture anchor.

FIG. 16 schematically illustrates an exemplary tissue fixation system.

### DETAILED DESCRIPTION

This disclosure is directed to tensionable tissue repair systems and surgical methods for repairing tissue defects within a joint. The system and methods described herein may be utilized to reduce, fixate, and compress tissue to bone. These and other features of this disclosure are described in further detail below.

An exemplary surgical method may include, inter alia, inserting a first suture anchor into a bone, passing a first suture of the first suture anchor through a tissue, feeding the first suture through a first attachment section of a surgical button, securing the first suture relative to the surgical button, inserting a second suture anchor into the bone at a position that is lateral to the first suture anchor, feeding a second suture of the second suture anchor through a second attachment section of the surgical button, and securing the second suture relative to the surgical button. The second attachment section is thinner and/or less dense than the first attachment section of the surgical button.

In any further embodiment, the second attachment section that is integrally formed with the first attachment section.

In any further embodiment, the first suture is passed through at least one of a first round hole or a second round hole formed through the first attachment section of the surgical button.

In any further embodiment, the second attachment section of the surgical button excludes any pre-formed openings.

In any further embodiment, the second attachment section is a tab-like projection that protrudes outwardly from the first attachment section.

In any further embodiment, securing the first suture relative to the surgical button includes splicing the first suture through itself to establish a mattress stitch that spans between a first opening and a second opening located within the first attachment section of the surgical button.

In any further embodiment, securing the first suture relative to the surgical button further includes tensioning the first suture to pull the tissue laterally over top of the first suture anchor.

In any further embodiment, securing the first suture relative to the surgical button further includes tensioning the first suture to fixate the surgical button against the tissue.

In any further embodiment, securing the second suture relative to the surgical button includes splicing the second suture through itself to establish a suture loop that is looped through the surgical button.

In any further embodiment, securing the second suture relative to the surgical button further includes tensioning the second suture to reduce the second suture further into place and provide a desired area of footprint compression over top of the tissue.

Another exemplary surgical method may include, inter alia, implanting a first suture anchor into a bone. A first suture of the first suture anchor extends through a tissue and is attached to a first attachment section of a surgical button. The surgical method further includes tensioning the first suture to fixate the surgical button against the tissue, inserting a second suture anchor into the bone at a position that is lateral to the first suture anchor, passing a second suture of the second suture anchor through a second attachment section of the surgical button, and securing the second suture back to the second suture anchor.

In any further embodiment, the second attachment section is a tab-like projection that protrudes outwardly from the first attachment section.

In any further embodiment, the second attachment section is thinner and/or less dense than the first attachment section of the surgical button.

In any further embodiment, tensioning the first suture includes splicing the first suture through itself to establish a mattress stitch that spans between a first opening and a second opening of the first attachment section of the surgical button.

In any further embodiment, tensioning the first suture pulls the tissue laterally over top of the first suture anchor.

In any further embodiment, securing the second suture back to the second suture anchor includes splicing the second suture through itself to establish a suture loop that is looped around the second attachment section of the surgical button.

In any further embodiment, securing the second suture back to the second suture anchor includes tensioning the second suture to reduce the second suture further into place and provide a desired area of footprint compression over top of the tissue.

In any further embodiment, the surgical method may include inserting a third suture anchor into the bone. A third suture of the third suture anchor extends through the tissue and is attached to a first attachment section of a second surgical button. The surgical method further includes tensioning the third suture to fixate the second surgical button against the tissue.

In any further embodiment, the surgical method may include inserting a fourth suture anchor into the bone at a position that is lateral to the third suture anchor, passing a fourth suture of the fourth suture anchor through the second surgical button, and securing the fourth suture back to the fourth suture anchor.

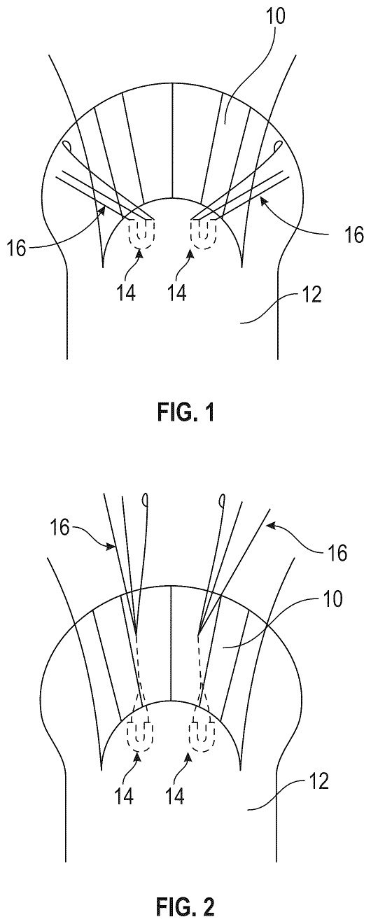

FIGS. 1-15 schematically illustrate various aspects associated with a surgical method for attaching a tissue 10 (e.g., ligament, tendon, graft, etc.) to a bone 12. The tissue 10 may have torn away from the bone 12 during vigorous exercise or sporting activities, for example. When such tears occur, reattachment is often necessary to repair the tissue defect. Although the surgical method is described herein for reapproximating and fixating torn tissue back to bone, the surgical methods of this disclosure could be utilized to repair any type of tissue effect.

Notably, the various figures accompanying this disclosure are not necessarily drawn to scale. Some features may be exaggerated or minimized to emphasize certain details of a particular component, system, or method.

The surgical method schematically illustrated in FIGS. 1-15 could be used in conjunction with a variety of orthopedic surgical repairs, including but not limited to rotator cuff repairs, Achilles tendon repairs, patellar tendon repairs, bicep repairs, ACL/PCL reconstructions, hip and shoulder reconstructions, among many others. The bone 12 may therefore be associated with any joint of the human musculoskeletal system (e.g., shoulder, knee, hip, ankle, etc.).

In an embodiment, the surgical method is performed as an arthroscopic procedure by working through various arthroscopic portals. However, the exemplary surgical method could alternatively be performed as an open procedure within the scope of this disclosure. As detailed below, the exemplary surgical method may be employed to reduce and then reattach the tissue 10 to the bone 12 in a manner that enhances footprint compression to maximize tissue-to-bone contact.

Referring first to FIG. 1, after appropriately preparing the bone 12 (e.g., by debriding, creating a bleeding bone bed, preparing bone sockets, etc.), a medial row of fixation devices may be implanted into the bone 12. The medial row of fixation devices may include one or more suture anchors 14. In an embodiment, the suture anchors 14 are knotless suture anchors that do not require the need to tie any knots in the various structures for reducing and securing the tissue 10 to the bone 12. However, traditional knot tying style suture anchors could alternatively or additionally be used in the medial row of fixation devices.

In an embodiment, the suture anchors 14 of the medial row are placed at the articular margin of the bone 12. However, other implantation locations could be selected based on the performing surgeon's own preferences. Notably, although two suture anchors 14 are illustrated as being part of the medial row in the illustrated embodiment, a greater or fewer number of suture anchors could be utilized as part of the surgical method within the scope of this disclosure. For example, the medial row could include but a single suture anchor 14.

Each suture anchor 14 may be pre-loaded with one or more sutures 16. The sutures 16 may include individual suture strands, multiple suture strands, suture tape, or any other suture-like product. As shown in FIG. 2, the sutures 16 may be passed upwardly through the tissue 10 after each suture anchor 14 is adequately fixated within the bone 12. At this stage of the surgical method, the tissue 10 is not directly over top of the suture anchors 14.

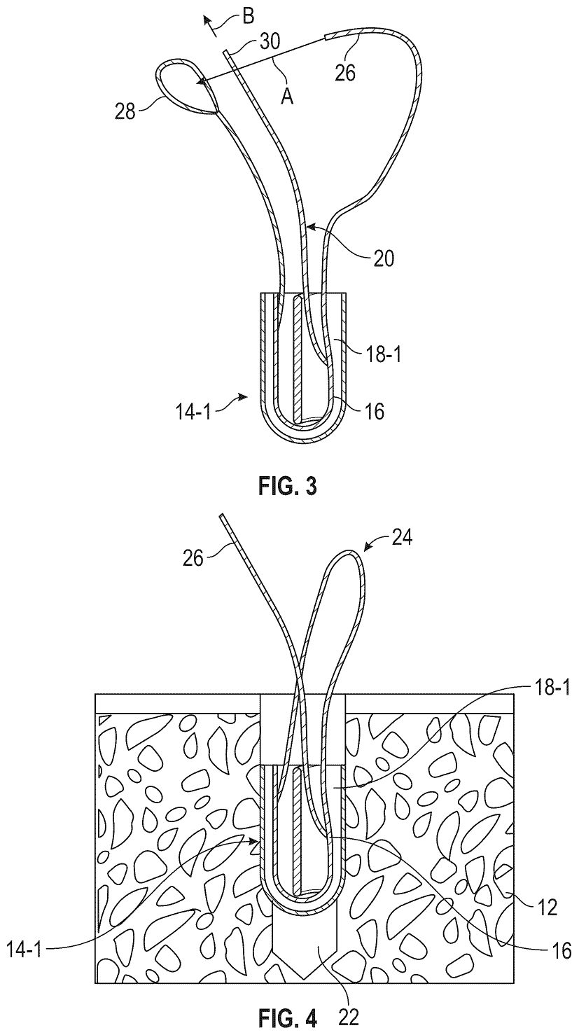

FIGS. 3-6 illustrate exemplary knotless suture anchors that can be utilized as the suture anchors 14 of the medial row of fixation devices when performing the surgical methods described herein. Knotless suture anchors similar to those shown in FIGS. 3-6 may be utilized either alone or in combination with one another as part of the medial row during the surgical method.

An exemplary knotless suture anchor 14-1 is illustrated in FIGS. 3 and 4. In this embodiment, the knotless suture anchor 14-1 is a “soft” anchor assembly made exclusively of soft, suture-based materials. The suture-based materials may include soft materials such as yarns, fibers, filaments, strings, fibrils, strands, sutures, etc., or any combination of such materials. The soft materials may be synthetic or natural materials, or combinations of synthetic and natural materials, and may be bio-degradable or non-degradable within the scope of this disclosure. The soft, suture-based materials confer the knotless suture anchor 14-1 with the ability to be inserted into bone sockets/holes and bunch together, collapse, expand and/or change shape to fixate within the socket/hole.

The knotless suture anchor 14-1 may include an anchor body 18-1 and a suture 16 received through the anchor body 18-1. A shuttle device 20 may be spliced through portions of the suture 16. The shuttle device 20 may be a passing wire or another suture, for example.

The anchor body 18-1 of the knotless suture anchor 14-1 may be inserted into a socket 22 formed in the bone 12 (see FIG. 4). The socket 22 may be a preformed opening formed in the bone 12 that is configured for receiving the anchor body 18-1.

The shuttle device 20 may be pre-assembled to the suture 16 as shown in FIG. 3, and the suture 16 may form a suture loop 24 after the suture 16 is shuttled through itself as shown in FIG. 4. For example, a suture tail 26 of the suture 16 may be passed through an eyelet 28 of the shuttle device 20 (in the direction of arrow A of FIG. 3), and then a free end 30 of the shuttle device 20 may be pulled (in the direction of arrow B of FIG. 3) to allow the suture 16 to pass through itself and form the suture loop 24. The perimeter of the suture loop 24 is adjustable to allow the construct to be self-cinching and to adjust the tension on the construct that is to be fixated.

In an exemplary embodiment, the suture anchors 14 of the medial row may include the design of the knotless suture anchor 14-1 for performing the surgical method steps herein. However, other type of suture anchors or combinations of suture anchors are contemplated within the scope of this disclosure.

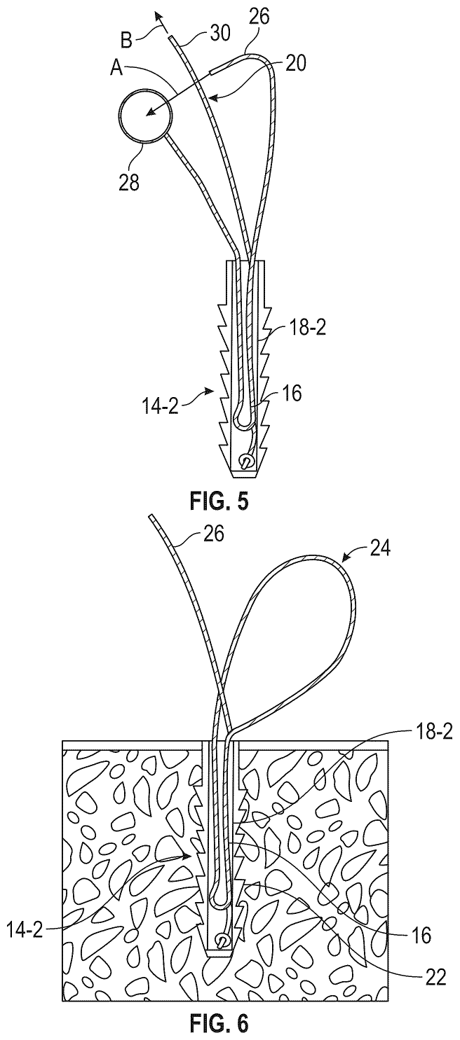

Another exemplary knotless suture anchor 14-2 is illustrated in FIGS. 5 and 6. The knotless suture anchor 14-2 may include an anchor body 18-2 and a suture 16 received through the anchor body 18-2. In this embodiment, the anchor body 18-2 is a relatively rigid plastic body and thus the knotless suture anchor 14-2 is not considered to be a “soft” anchor assembly.

The knotless suture anchor 14-2 may include a tensionable knotless mechanism that is similar to that of the knotless suture anchor 14-1. For example, a shuttle device 20 may be spliced through portions of the suture 16. The shuttle device 20 may be a passing wire or another suture, for example. The anchor body 18-2 of the knotless suture anchor 14-2 may be inserted into a socket 22 formed in the bone 12 (see FIG. 6). The socket 22 may be a preformed opening formed in the bone 12 that is configured for receiving the anchor body 18-2. The shuttle device 20 may be pre-assembled to the suture 16 as shown in FIG. 5 and may be utilized to form a suture loop 24 after the suture 16 is shuttled through itself as shown in FIG. 6. For example, a suture tail 26 of the suture 16 may be passed through an eyelet 28 of the shuttle device 20 (in the direction of arrow A of FIG. 5), and then a free end 30 of the shuttle device 20 may be pulled (in the direction of arrow B of FIG. 5) to allow the suture 16 to pass through itself and form the suture loop 24. The perimeter of suture loop 24 is adjustable to allow the construct to be self-cinching and to adjust the tension on the construct to be fixated.

In an exemplary embodiment, the suture anchors 14 of the medial row may include the design of the knotless suture anchor 14-2 (rather than that of the knotless suture anchor 14-1, for example) for performing the surgical method steps described herein. However, other type of suture anchors or combinations of suture anchors are contemplated within the scope of this disclosure.

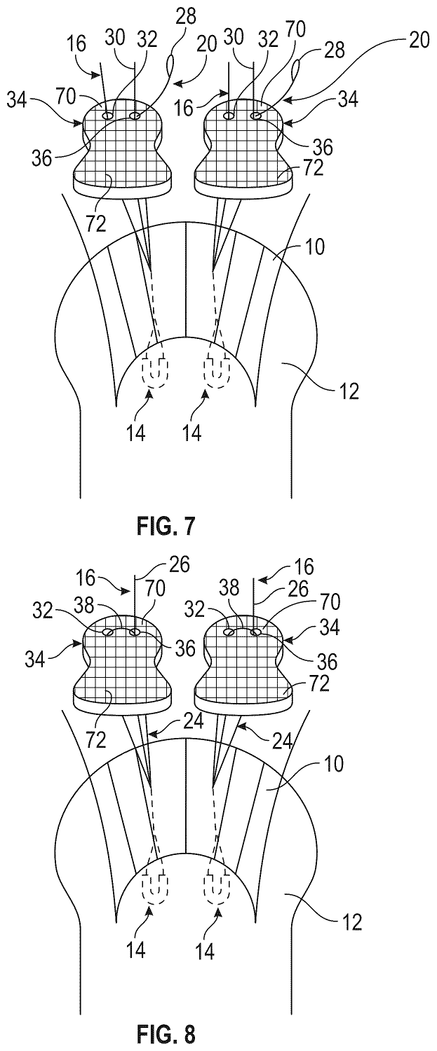

Referring now to FIG. 7, the surgical method depicted in FIGS. 1 and 2 may continue by feeding both the suture 16 and the shuttle device 20 of each suture anchor 14 through a surgical button 34. In general, the number of surgical buttons 34 utilized during the surgical method will match the number of suture anchors 14 utilized as part of the medial row. Each surgical button 34 may be at least partially made of a passable material that can allow sutures or other surgical devices to be passed directly through the surgical button 34. The surgical buttons 34 may be embroidered or woven from polyester or some other permanent or absorbable materials, molded/machined/3D printed from a tissue compatible polymer, etc.

In an embodiment, each surgical button 34 includes a first attachment section 70 and a second attachment section 72 that is integrally formed with the first attachment section 70. The first attachment section 70 may include a generally round or oval shape, and the second attachment section 72 may be a tab-like projection that protrudes outwardly from the first attachment section 70. The first attachment section 70 and the second attachment section 72 may have different structural properties. For example, the second attachment section 72 may be thinner and/or less dense than the first attachment section 72. It is therefore generally easier in some implementations to pass suture or other structures through the second attachment section 72 as compared to the first attachment section 70. However, other configurations of the surgical buttons 34 are contemplated within the scope of this disclosure.

A first opening 32 and a second opening 36 may optionally be formed through the first attachment section 70 of each surgical button 34. The first opening 32 and the second opening 36 may be configured for receiving suture. For example, as shown in FIG. 7, the suture 16 of each suture anchor 14 may be fed through the first opening 32 of the surgical button 34, and the shuttle device 20 (both free end 30 and eyelet 28, for example) may be fed through the second opening 36 of the surgical button 34. At this point, the suture 16 and the shuttle device 20 are considered provisionally connected to the first attachment section 70 of the surgical button 34.

In an embodiment, the first opening 32 and the second opening 36 are round holes formed through the surgical button 34. However, other configurations are contemplated within the scope of this disclosure.

Referring now to FIG. 8, each suture 16 may then be spliced through itself using the shuttle device 20 to form the suture loop 24 about the surgical button 34. Splicing the suture 16 through itself in this manner creates a mattress stitch 38 that spans between the first opening 32 and the second opening 36 of the surgical button 34. The mattress stitch 38 may be established by an apex of the suture loop 24, for example.

In an alternative embodiment, the surgical button 34 may be preconnected to the suture anchor 14 by the spliced suture 16, and the surgical steps shown in FIGS. 1, 2, 7, and 8 can be essentially combined together by utilizing a transtendinous insertion of the suture anchor 14. During such a transtendinous approach, the suture anchor 14 may be inserted directly through the tissue 10 and into the underlying bone 12 without preforming holes in the bone 12.

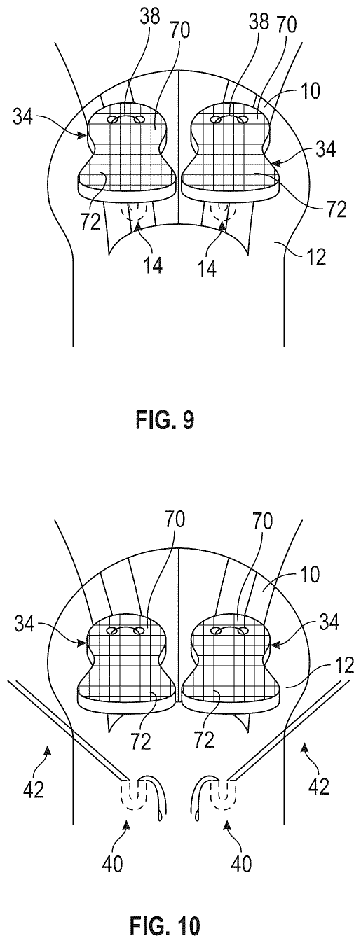

The sutures 16 may be tensioned to approximate each surgical button 34 firmly against the tissue 10 (see FIG. 9). The tissue 10 therefore reduces into place over top of the suture anchors 14 (e.g., is pulled laterally) and is fixated against the bone 12. The suture tails 26 of the sutures 16 may be removed (e.g., cut) once tensioning is complete. At this stage of the surgical method, the suture anchors 14 are located underneath the tissue 10 and are at least partially underneath the surgical buttons 34.

The surgical repair can be considered complete at this point of the surgical method. Alternatively, if double row fixation is desired, the surgical method can proceed by implanting a lateral row of fixation devices into the bone 12. As shown in FIG. 10, the lateral row of fixation devices may include one or more suture anchors 40. In an embodiment, the suture anchors 40 are knotless suture anchors that do not require the need to tie any knots in the various structures for achieving fixation. However, traditional knot tying style suture anchors could alternatively or additionally be used in the lateral row.

In an embodiment, the suture anchors 40 of the lateral row are placed laterally from the medial row of suture anchors 14 and slightly distal to the greater tuberosity of the bone 12. However, other implantation locations could be selected based on the performing surgeon's own preferences and depending on the type of orthopedic procedure being performed. Notably, although two suture anchors 40 are illustrated as being part of the lateral row in the illustrated embodiment, a greater or fewer number of suture anchors could be utilized as part of the surgical method within the scope of this disclosure.

Each suture anchor 40 may be pre-loaded with one or more sutures 42. The sutures 42 may include individual suture strands, multiple suture strands, suture tape, or any other suture-like product.

The suture anchors 40 of the lateral row may include the same or a different design as the suture anchors 14 of the medial row. In an embodiment, the suture anchors 40 of the lateral row may include the design of the knotless suture anchor 14-1. In another embodiment, the suture anchors 40 of the lateral row may include the design of the knotless suture anchor 14-2.

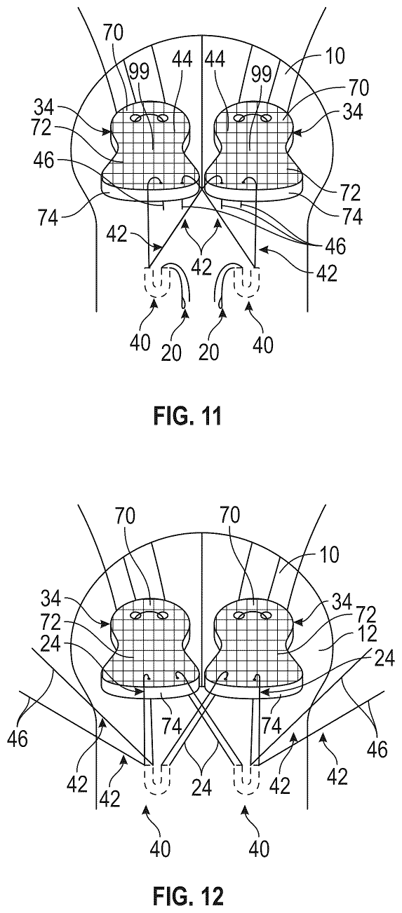

Referring now to FIG. 11, one suture 42 from each suture anchor 40 may be fed (e.g., from top to bottom) through the second attachment section 72 of each surgical button 34. Due to the thinner/less dense configuration of the second attachment section 72, pre-formed openings are generally not required in the second attachment section 72 for accommodating the sutures 42 and therefore may be excluded within the second attachment section 72. The sutures 42 may be fed through the second attachment section 72 either before or after fully approximating the surgical buttons 34 firmly down against the tissue 10. The sutures 42 may be positioned such that a suture tail 46 of each suture 42 is looped over a peripheral portion 74 of the second attachment section 72.

Referring next to FIGS. 11 and 12, each suture 42 may be secured back to its suture anchor 40 by splicing the suture 42 through itself using the shuttle device 20. The splicing procedure forms the suture loop 24 about the surgical button 34, thereby securing each suture 42 to the surgical button 34. The suture tails 46 may be further tensioned to reduce the sutures 42 further into place and more firmly fixate the tissue 10 against the bone 12. The suture tails 46 of the sutures 16 may be removed (e.g., cut) once tensioning is complete.

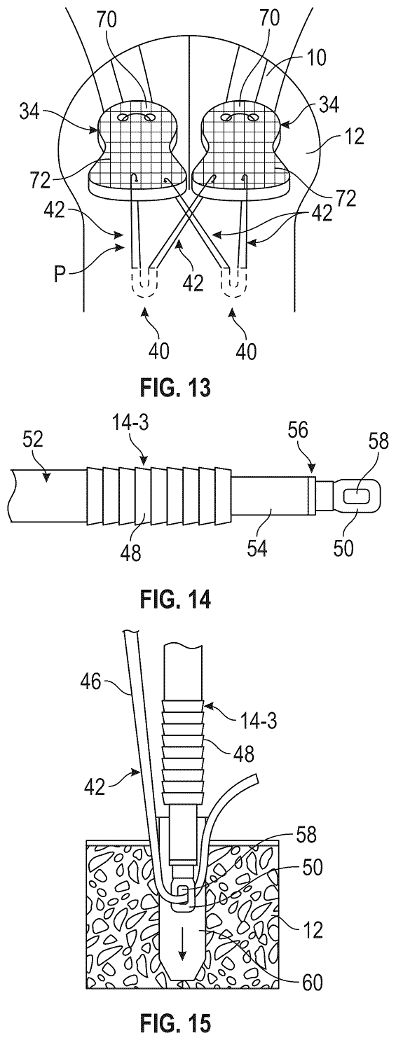

The final construct achieved by the surgical method is shown in FIG. 13. The sutures 42 may be arranged in various bridging configurations between the second attachment sections 72 of the surgical buttons 34 and the suture anchors 40 of the lateral row. For example, as shown in FIG. 13, the sutures 42 may be arranged in a crisscross pattern P that provides a desired area of footprint compression over top of the tissue 10. Any bridging configuration using any number of suture anchors, surgical buttons, and sutures can be achieved as part of the surgical method.

In the above embodiments, the sutures 42 are connected to the surgical buttons 34 after implanting the lateral row suture anchors 40 into the bone 12. However, other implementations are possible. For example, the suture anchors 40 could be implanted after connecting the sutures 42 to the surgical buttons 34 by employing the design of another exemplary knotless suture anchor 14-3 shown in FIGS. 14-15.

The knotless suture anchor 14-3 may include an anchor body 48 and an eyelet 50. In this embodiment, the anchor body 48 and the eyelet 50 are relatively rigid plastic structures and thus the knotless suture anchor 14-3 is not considered to be a “soft” anchor assembly.

The anchor body 48 may be pre-loaded onto a driver 52. The anchor body 48 may be configured as a screw or an interference plug that is appropriately cannulated for receiving a shaft 54 of the driver 52. The eyelet 50 may be provided at a distal end 56 of the driver 52. The eyelet 50 may be releasably attached to the distal end 56 and may include an aperture 58 for receiving one or more sutures.

The anchor body 48 and the eyelet 50 of the knotless suture anchor 14-3 may be inserted into a socket 60 formed in the bone 12 (see FIG. 15). The socket 60 may be a preformed opening formed in the bone 12 that is configured for receiving the anchor body 48 and the eyelet 50. One of the suture tails 46 of the sutures 42 may be loaded through the eyelet 50, and then the eyelet 50 may be inserted into the socket 60. The suture tail 46 may then be tensioned prior to moving the anchor body 48 down toward the eyelet 50 within the socket 60. Once implanted within the socket 60, the anchor body 48 may trap the suture 42 between the bone 12 and the anchor body 48 in order to fixate the suture 42 in place. The knotless suture anchor 14-3 therefore provides the ability to implant the lateral row suture anchors 40 after connecting the sutures 42 to the surgical buttons 34 during the surgical method.

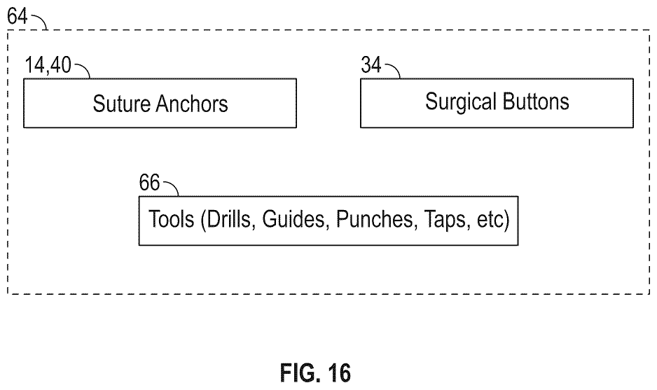

FIG. 16 schematically illustrates an exemplary tissue fixation system 64 that may be provided for performing the surgical method described above. The tissue fixation system 64 may be provided in the form of a surgical kit that includes all the necessary tools and components for performing surgical methods for reducing and reattaching torn tissue to bone. In an embodiment, the tissue fixation system 64 may include at least the following components:
1. At least (4) suture anchors 14, 40;
2. At least two surgical buttons 34; and
3. Various tools 66 (e.g., disposable drills, drill guides, punches, taps, etc.) for inserting the suture anchors 14, 40.

Other components or different combinations of components could be provided as part of the tissue fixation system 64 within the scope of this disclosure. For example, the tissue fixation system 64 could include various templates, scorers, curettes, and/or measuring devices that may be utilized to help prepare the tissue 10 and the bone 12 for performing the surgical methods discussed herein.

The tissue fixation systems and surgical methods described herein may be utilized to approximate, fixate, and compress tissue to bone. The proposed systems and methods provide a multi-point fixation configuration for fixating tissue to bone. The use of surgical buttons in combination with medial and lateral suture anchors provides a relatively stiff construct for maintaining footprint compression, thereby maximizing tissue-to-bone contact.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A surgical method, comprising:
inserting a first suture anchor into a bone;
passing a first suture of the first suture anchor through a tissue;
feeding the first suture through a first attachment section of a surgical button;
securing the first suture relative to the surgical button, inserting a second suture anchor into the bone at a position that is lateral to the first suture anchor;
feeding a second suture of the second suture anchor through a second attachment section of the surgical button, wherein the second attachment section is thinner and/or less dense than the first attachment section of the surgical button; and
securing the second suture relative to the surgical button.

2. The surgical method as recited in claim 1, wherein the second attachment section is integrally formed with the first attachment section.

3. The surgical method as recited in claim 2, wherein the first suture is passed through at least one of a first round hole or a second round hole formed through the first attachment section of the surgical button.

4. The surgical method as recited in claim 3, wherein the second attachment section excludes pre-formed openings.

5. The surgical method as recited in claim 2, wherein the second attachment section is a tab-like projection that protrudes outwardly from the first attachment section.

6. The surgical method as recited in claim 1, wherein securing the first suture relative to the surgical button includes:
splicing the first suture through itself to establish a mattress stitch that spans between a first opening and a second opening located within a first attachment section of the surgical button.

7. The surgical method as recited in claim 6, wherein securing the first suture relative to the surgical button further includes:
tensioning the first suture to pull the tissue laterally over top of the first suture anchor.

8. The surgical method as recited in claim 6, wherein securing the first suture relative to the surgical button further includes:
tensioning the first suture to fixate the surgical button against the tissue.

9. The surgical method as recited in claim 1, wherein securing the second suture relative to the surgical button includes:
splicing the second suture through itself to establish a suture loop that is looped through the surgical button.

10. The surgical method as recited in claim 9, wherein securing the second suture relative to the surgical button further includes:
tensioning the second suture to reduce the second suture further into place and provide a desired area of footprint compression over top of the tissue.

11. A surgical method, comprising:
implanting a first suture anchor into a bone, wherein a first suture of the first suture anchor extends through a tissue and is attached to a first attachment section of a surgical button;
tensioning the first suture to fixate the surgical button against the tissue,
inserting a second suture anchor into the bone at a position that is lateral to the first suture anchor;
passing a second suture of the second suture anchor through a second attachment section of the surgical button; and
securing the second suture back to the second suture anchor.

12. The surgical method as recited in claim 11, wherein the second attachment section is a tab-like projection that protrudes outwardly from the first attachment section.

13. The surgical method as recited in claim 12, wherein the second attachment section is thinner and/or less dense than the first attachment section of the surgical button.

14. The surgical method as recited in claim 11, wherein tensioning the first suture includes:
splicing the first suture through itself to establish a mattress stitch that spans between a first opening and a second opening of the first attachment section of the surgical button.

15. The surgical method as recited in claim 11, wherein tensioning the first suture fixates the surgical button over top of the first suture anchor.

16. The surgical method as recited in claim 11, wherein securing the second suture back to the second suture anchor includes:
splicing the second suture through itself to establish a suture loop that is looped around the second attachment section of the surgical button.

17. The surgical method as recited in claim 16, wherein securing the second suture back to the second suture anchor includes:
tensioning the second suture to reduce the second suture further into place and provide a desired area of footprint compression over top of the tissue.

18. The surgical method as recited in claim 11, comprising:
implanting a third suture anchor into the bone, wherein a third suture of the third suture anchor extends through the tissue and is attached to a first attachment section of a second surgical button; and
tensioning the third suture to fixate the second surgical button against the tissue.

19. The surgical method as recited in claim 18, comprising:
inserting a fourth suture anchor into the bone at a position that is lateral to the third suture anchor;
passing a fourth suture of the fourth suture anchor through the second surgical button; and
securing the fourth suture back to the fourth suture anchor.
